(19) United States
(12) Patent Application Publication
(10) Pub. No.: US 2017/0020534 A1
(43) Pub. Date: Jan. 26, 2017

(54) FLUTED BONE AWL AND METHOD OF USE

(71) Applicant: DePuy Mitek, LLC, Raynham, MA (US)

(72) Inventor: Daniel Gamache, Fall River, MA (US)

(21) Appl. No.: 15/283,688

(22) Filed: Oct. 3, 2016

Related U.S. Application Data
(63) Continuation of application No. 12/823,490, filed on Jun. 25, 2010, now abandoned.

(51) Int. Cl. A61B 17/16; A61B 50/30

(52) U.S. Cl. CPC A61B 17/1615; A61B 50/30

(57) ABSTRACT

An awl provides for creating an accurate hole in a bone, such as for implantation of a suture anchor. The awl comprises an elongated shaft having a distal tip which terminates in a sharp point adapted to penetrate bone. At least one cutting flute is provided on the shaft and has a longitudinally extending cutting edge formed on the shaft proximal to and adjacent to the sharp point whereby to allow removal of bone via the cutting flute upon rotation of the shaft about a central longitudinal axis thereof inside of the bone.

### BACKGROUND

The present application relates to tools and methods for creating a bone hole for implantation of a medical device such as a suture anchor.

Many suture anchors including many screw-threaded anchors, are designed for insertion into a hole previously prepared in bone. The hole can be prepared using a powered drill or other rotary tool with a properly sized drill bit. More simply, and ideally more quickly for the surgeon, a manual tool such as an awl (a distally pointed tool typically provided with a proximal handle) can be used to prepare the hole. Awls necessarily increase in diameter proximally from the pointed tip, and the amount of axial force required to insert the awl to a desired depth into bone, to achieve a hole having a diameter appropriate to receive a given anchor, increases nonlinearly and not necessarily predictably to the desired diameter.

In addition, as the awl is inserted axially past its tip to enlarge the hole, the surrounding bone, particularly the relatively hard cortical (outer) bone layer, becomes compressed. Not only does this compression contribute to the difficulty of further inserting the awl into the bone, but the compressed bone requires additional force to displace, compared with uncompressed bone, making it more difficult for the surgeon to control completion of the hole to the correct diameter with precision, increasing the probability of damaging (e.g., micro-fracturing) the surrounding bone due to the larger forces required, increasing the force required to remove the awl from the freshly prepared hole, and making it more difficult (requiring excessive torque) to thread the suture anchor into the compressed cortical bone when the hole preparation is complete.

An alternative to using a manual awl alone to prepare the anchor hole is to use an awl to start the hole, then to use a powered or hand-operated reamer to increase the hole diameter appropriate to the anchor diameter, while removing most of the compressed cortical bone with the flutes of the reamer. This extra step increases procedural complexity, offsetting a primary advantage of using an awl to prepare the bone hole.

It would be advantageous to have a single manual tool for quickly and reproducibly preparing bone holes for receiving threaded suture anchors or other threaded devices.

### SUMMARY OF THE INVENTION

An awl according to the present invention provides for creating a hole in a bone, such as for implantation of a suture anchor. The awl comprises an elongated shaft having a distal tip which terminates in a sharp point adapted to penetrate bone. At least one cutting flute is provided on the shaft and has a longitudinally extending cutting edge formed on the shaft proximal to and adjacent to the sharp point whereby to allow removal of bone via the cutting flute upon rotation of the shaft about a central longitudinal axis thereof inside of the bone.

Preferably, the shaft has a major radius from the central longitudinal axis at the cutting edge and a minor radius from the central longitudinal axis circumferentially away from the cutting edge, the major radius having a magnitude exceeding a magnitude of the minor radius. These are measured at a similar longitudinal distance along the shaft.

In an awl having a plurality of cutting flutes with cutting edges, the shaft at a longitudinal extent within the cutting flute has a major radius from the central longitudinal axis to each cutting edge and a minor radius from the central longitudinal axis to an exterior surface of the shaft between adjacent cutting flutes. The minor radius has a magnitude less than a magnitude of the major radius.

Preferably the distal tip has a first more aggressive taper towards the distal tip and a fluted section proximal of the distal tip, defined by the at least one flute, has a gentler taper, preferably equivalent to a reduction in diameter of 20 to 40 thousands of an inch over a length of 0.75 inches.

Preferably the awl has a plurality of cutting flutes, and more preferably two to four cutting flutes.

Preferably, the shaft is formed of a biocompatible material, is sterile and is packaged in a bacteria proof sterile package.

Preferably, the awl has depth indicia on the shaft whereby to allow a user to gauge a depth of a hole created with the awl.

In one aspect of the invention, the awl is packaged with instructions for using comprising the following steps: driving the distal tip of the awl into the bone to initiate creation of a hole therein; and rotating the shaft to create the hole with a predetermined diameter.

A method for implanting a device into a bone according to the present invention comprises the steps of: driving a sharp distal tip of an awl into the bone to initiate creation of a hole therein; with at least one cutting flute on a shaft of the awl proximal to and adjacent to the sharp distal tip, removing bone from about the hole by rotating the shaft about a central longitudinal axis thereof to create the hole with a predetermined diameter; and implanting the device into the hole.

Preferably, the shaft has a major radius at a cutting edge on the flute and a minor radius at a point circumferentially away from the cutting edge, which minor radius has a magnitude less than the major radius, wherein as the shaft is rotated to remove bone, the shaft at the minor radius engages the bone with less outward radial force than at the major diameter or fails to engage the bone.

Preferably, the awl is pushed to a predetermined depth into the bone using a depth indicia on the shaft, which pushing is preferably accomplished concurrently with the rotation.

### BRIEF DESCRIPTION OF THE DRAWINGS

FIG. 1 is a perspective view of a fluted awl according to the present invention;

FIG. 2 is a cross-sectional view of the flutes of the awl taken along lines 2-2 of FIG. 1;

FIGS. 3A to 3F are end views of alternative version of the awl flutes according to the present invention;

FIGS. 4A to 4F are side elevation views of the fluted portions of the corresponding awls of FIGS. 3A to 3F;

FIG. 5 is an exploded perspective view of the awl of FIG. 1 in its packaging; and

FIGS. 6A to 6C are side elevations in cut-away of the awl of FIG. 1 creating a hole in bone and the implantation of a suture anchor therein.

### DETAILED DESCRIPTION

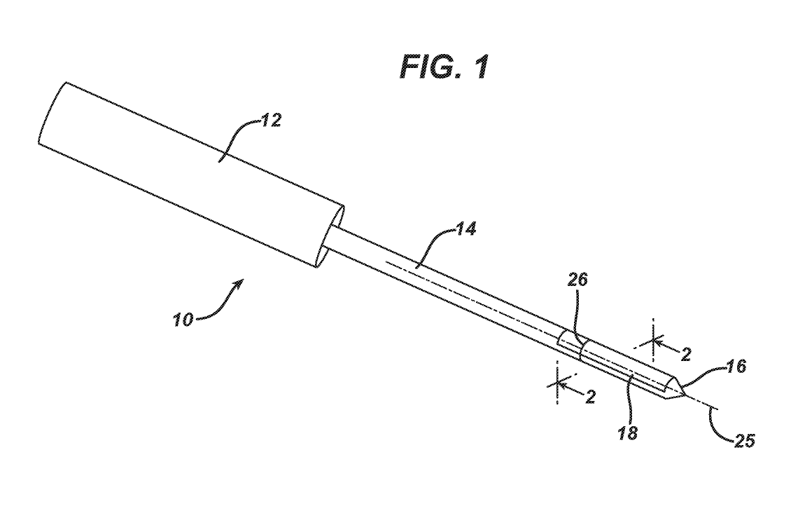

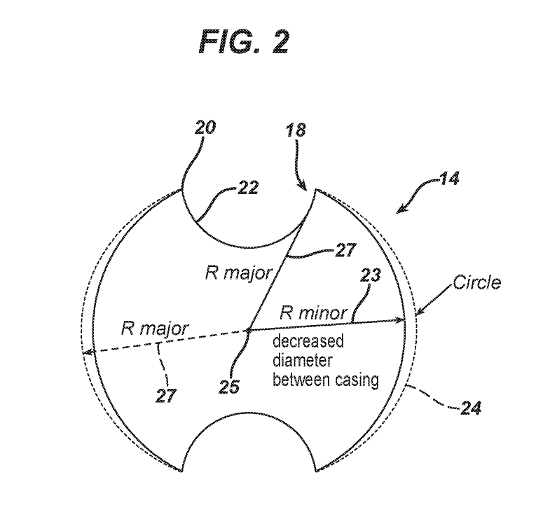

FIGS. 1 and 2 illustrates a fluted awl 10 according to the present invention. It comprises a proximal handle 12 having an elongated shaft 14 extending distally, axially therefrom. The shaft 14 terminates in a sharp distal tip 16. Immediately proximal of the tip 16 the shaft 14 bears a plurality of axial cutting flutes 18 formed therein. Each flute 18 comprises an axially oriented sharp cutting edge 20 and an adjacent undercut portion 22. The cutting flutes 18 allow cutting of bone (not shown in FIG. 1) without unduly compressing or stressing the remaining bone material.

At the flutes 18, the shaft 14 has a diameter that tapers gently toward the distal tip 16, although a constant diameter is possible with the present invention. Preferably, this section tapers 20 to 40 thousandths over a length of flutes of 0.75 inches, and preferably tapers at about 30 thousandths. Higher degrees of taper are more appropriate for softer bone and lower degrees of taper are more appropriate for harder bone. Although generally round in cross section a minor radius 23 from a central longitudinal axis 25 of the shaft 14 along the circumference away from the cutting edges 20 can be slightly decreased from a major radius 27 of a perfect circle 24 which intersects the cutting edges 20 so that that portion of the shaft 14 will not compress the surrounding bone (not shown in FIG. 2), not drag during rotation and to facilitate removal of the shaft from the bone after completion. Preferably, at its minimum the diameter is reduced 1 to 2 percent from circular with reductions of up to 20% being useful in softer bone. A depth indication line 26 is preferably provided to show a surgeon when proper depth has been achieved.

The fluted awl 10 is shown with two flutes 18 each having two cutting edges 20 to allow cutting in both clockwise and counterclockwise rotation. Other configurations are contemplated within the scope of the present invention. For instance, rather than extending purely axially, the cutting flutes 18 could be provided in a spiral pattern, but the straight axial orientation is preferred as it will not induce axial movement of the shaft 14 as it is rotated.

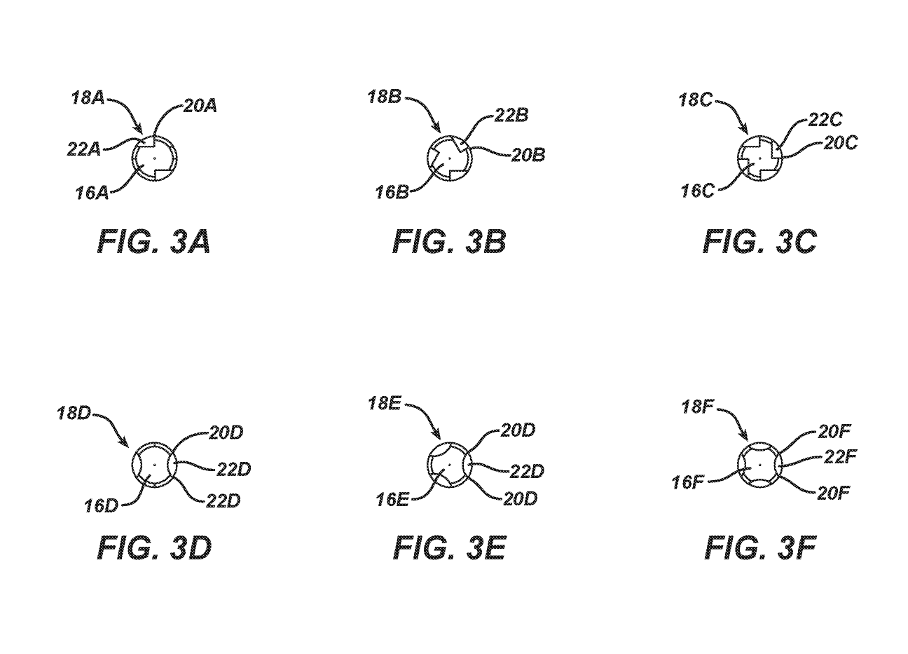

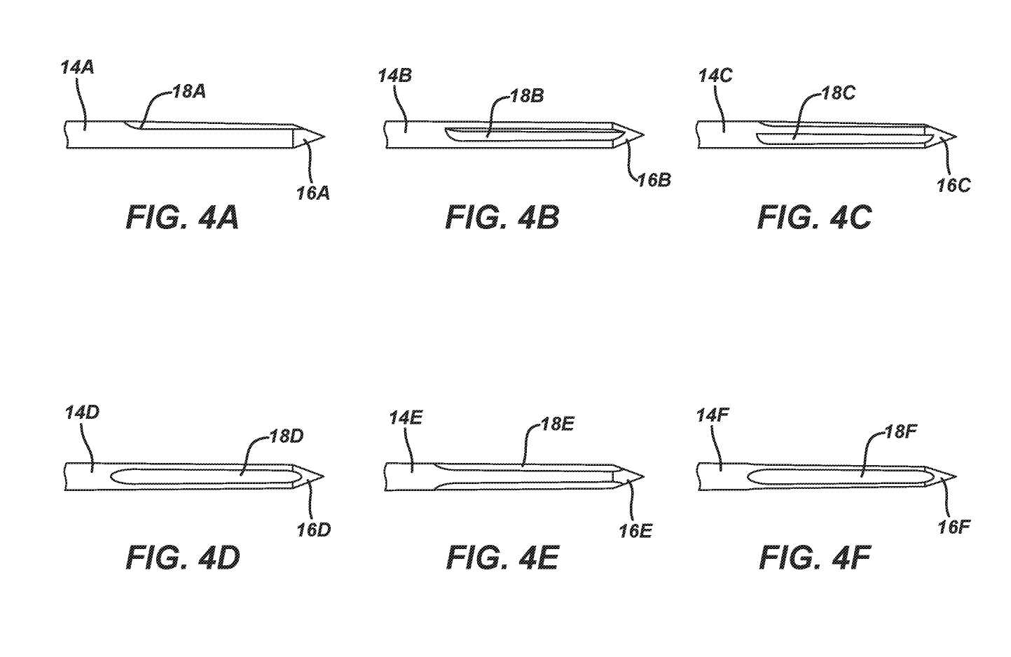

FIGS. 3A to F show end views and FIGS. 4A to F side views of corresponding alternative embodiments of a fluted awl according to the present invention with like part numbers to those of awl 10 being the same with the addition of the corresponding figure sub letter. FIGS. 3A and 4A show a shaft 14A having two flutes 18A, each having a radial cutting edge 20A. FIGS. 3B and 4B show three flutes 18B, each having a radial cutting edge 20B. FIGS. 3C and 4C show four flutes 18C, each having a radial cutting edge 20C. FIGS. 3D and 4D show a shaft 14D having two flutes 18D, each of which has two cutting edges 20D, allowing cutting in both clockwise and counterclockwise rotation. FIGS. 3E and 4E show three similar bi-directional flutes 18E and FIGS. 3F and 4F show four bi-directional flutes 18F.

The awl 10 is formed from biocompatible materials. The shaft is preferable formed of an appropriate surgical metal such as 630 grade 17-4 stainless steel heat treated to H900. The handle may be formed from any appropriate surgical instrument handle material, although stainless steel is preferred if the instrument is to be reusable and subject to numerous cleaning and sterilization procedures.

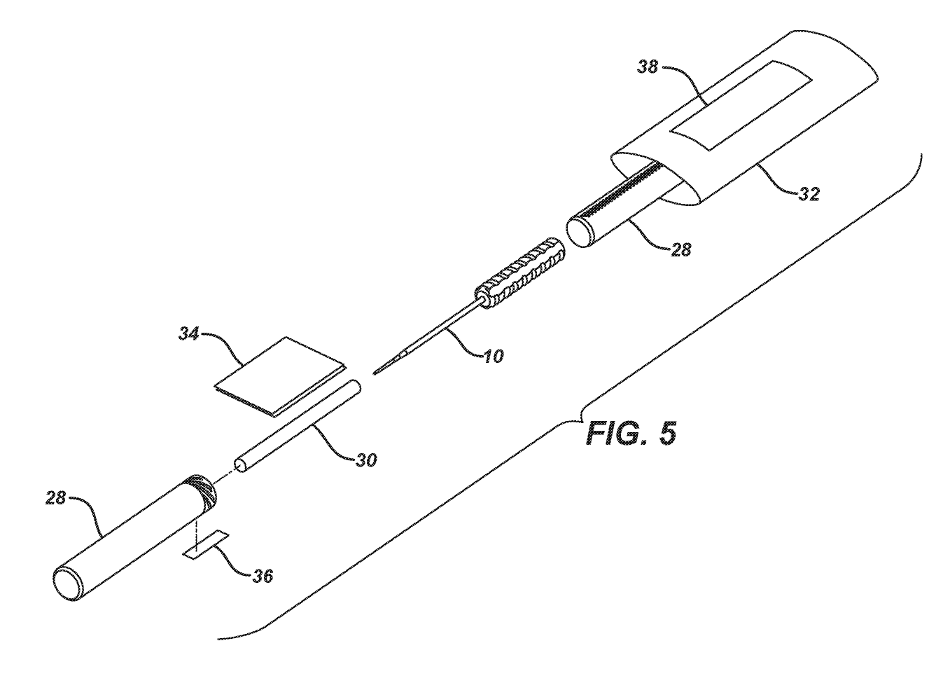

Preferably, the awl 10 is provided sterile and packaged for sterile removal and use. FIG. 5 shows the awl 10 packaged within a two piece sterile twist tube 28 and having a sterile protective closed end tubular guard 30 over the flutes 18 and distal tip 16. This is in turn packaged within a sterile envelope 32 which includes instructions for use 34 therein. Tamper evidence tape 36 is provided on the twist tube 28. A label 38 on the envelope 32 provides instructions for sterile removal of the awl 10. A clean but not necessarily sterile person opens the envelope 32 allowing a sterile person to remove the sterile twist tube 28 from the envelope 32 while maintaining the sterility of the twist tube 28 which is then opened within a sterile field.

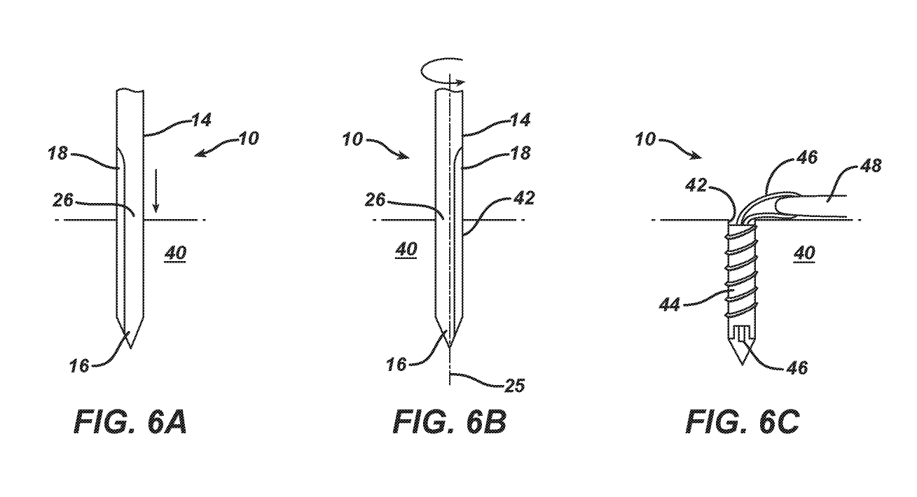

FIGS. 6A to 6C illustrate use of the awl 10. After the awl 10 is removed from its sterile packaging 32 and 28 it is placed against a bone 40 at the desired location and is pushed or malleted in as shown in FIG. 6A. It is then rotated, as shown in FIG. 6B to cut away the compressed bone and to create a hole 42 of appropriate diameter. The awl is also pressed in during the rotation to bring it to the proper depth as shown by the depth indicator 26. Then the awl 10 is removed and a suture anchor 44 is inserted into the hole 42 and suture 46 attached thereto is used such as for attaching soft tissue 48 to the bone 40 as shown in FIG. 6C.

The invention has been described with reference to the preferred embodiments. Obviously, modifications and alterations will occur to others upon reading and understanding the preceding detailed description. It is intended that the invention be construed as including all such modifications and alterations insofar as they come within the scope of the appended claims or the equivalents thereof.

1-13. (canceled)

14. An awl for creating a hole in bone, the awl comprising:
an elongated shaft having a distal tip;
the distal tip having a sharp point adapted to penetrate bone;
a pair of symmetrical cutting flutes on shaft;
a first one of the pair of cutting flutes being oriented to cut during rotation of the shaft in a first direction and having a first length and first axial location along the shaft with respect to the distal tip; and
a second one of the pair of cutting flutes being oriented to cut during rotation of the shaft in a second direction opposite to the first direction, and having a second length and second axial location along the shaft with respect to the distal tip, the first length and second length being equal and the first axial location and second axial location being equal.

15. An awl according to claim 14 and further comprising a second pair of symmetrical cutting flutes.

16. An awl according to claim 14 and further comprising a depth indicia on the shaft whereby to allow a user to gauge a depth of the hole created by the awl.
